(19) United States
(12) Patent Application Publication
(10) Pub. No.: US 2023/0310674 A1
(43) Pub. Date: Oct. 5, 2023

(54) LIGHT IRRADIATION SYSTEM

(71) Applicant: NIPPON TELEGRAPH AND TELEPHONE CORPORATION, Tokyo (JP)

(72) Inventors: Tomohiro TANIGUCHI, Musashino-shi, Tokyo (JP); Ayako IWAKI, Musashino-shi, Tokyo (JP); Kazuhide NAKAJIMA, Musashino-shi, Tokyo (JP); Nobutomo HANZAWA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Toyo (JP); Kazutaka HARA, Musashino-shi, Tokyo (JP); Atsuko KAWAKITA, Musashino-shi, Tokyo (JP)

(73) Assignee: NIPPON TELEGRAPH AND TELEPHONE CORPORATION, Tokyo (JP)

(21) Appl. No.: 18/015,685

(22) PCT Filed: Nov. 2, 2020
(86) PCT No.: PCT/JP2020/041092

§ 371 Date: Jan. 11, 2023

(30) Foreign Application Priority Data
Jul. 17, 2020 (WO) PCT/JP2020/027836

(51) Int. Cl. A61L 2/10; A61L 2/24; G02F 1/37

(52) U.S. Cl. CPC A61L 2/10; A61L 2/24; G02F 1/37; A61L 2202/11

(57) ABSTRACT

An object of the present invention is to provide a light irradiation system capable of securing economic efficiency, flexibility, reliability, and safety, and further capable of reducing a transmission loss between a center side and a remote side.

The present light irradiation system can secure economic efficiency by sharing a single light source installed on the center side with a plurality of irradiation places. The present light irradiation system can irradiate a place to be sterilized with ultraviolet light output by moving the optical fiber tip end on the remote side in a pinpoint manner, and can also secure flexibility. In addition, the present light irradiation system can secure reliability and safety by performing output control of the light source on the center side. Furthermore, in the present light irradiation system, light in a low-loss wavelength region is transmitted from the center side through the optical fiber, wavelength conversion is performed on the remote side, and thus the transmission loss between the center side and the remote side can be reduced.

### TECHNICAL FIELD

The present disclosure relates to a light irradiation system that propagates light from a light source through an optical fiber and irradiates a remote portion with the light.

### BACKGROUND ART

A new type of coronavirus (2019-nCoV) infection reported to have arisen in December 2019 has spread worldwide, and keen interest in prevention of infection is expected to increase in the future. One infection prevention measure is “sterilization” involving killing bacteria, and this can be further divided into “disinfection” of killing pathogenic bacteria to the extent that infection can be prevented, and “sterilization” of completely killing and removing microorganisms by treatment with high-pressure water vapor or the like. In the Japanese Pharmacopoeia, when the survival probability of a microorganism is 1 in a million or less, the microorganism is defined as having been sterilized, but the target is a bacterium not on a living body but mainly on a tool or the like.

On the other hand, disinfection methods are divided into a chemical disinfection method of sterilizing pathogenic microorganisms using a disinfectant and a physical disinfection method of not using a disinfectant. Further, physical disinfection methods are divided into boiling disinfection, hot water disinfection, steam disinfection, intermittent disinfection, and ultraviolet light sterilization.

Among these, ultraviolet light sterilization is sterilization through irradiation of light in a wavelength band around 260 nm, and it is known that, by irradiating bacteria or pathogenic bacteria with light in this wavelength range, DNA in the bacteria and pathogenic bacteria cause a photochemical reaction such as a hydration phenomenon, dimer formation, and decomposition, and as a result, bacteria are killed and inactivated. As a light source for ultraviolet light sterilization, a mercury lamp having a spectrum of 254 nm has been mainly used so far, but research and development of a small and highly efficient deep ultraviolet LED as an alternative device have been actively conducted due to environmental considerations, and a highly functional sterilization robot equipped with these light sources, a stationary air purifier, a low-cost portable sterilizer for consumers, and the like are commercially available as products (for example, refer to Non Patent Literature 1 to 3).

### CITATION LIST

### Non Patent Literature

Non Patent Literature 1: Website of Kantum Ushikata Co., Ltd.
(https://www.kantum.co.jp/product/sakkin_robot/sakkinn_robo t/UVDrobot) retrieved on Jun. 26, 2020
Non Patent Literature 2: Website of Iwasaki Electric Co., Ltd.
(https://www.iwasaki.co.jp/optics/sterilization/air/air03.h tml) retrieved on Jun. 26, 2020
Non Patent Literature 3: Website of Funakoshi Co., Ltd.
(https://www.funakoshi.co.jp/contents/68182) retrieved on Jun. 26, 2020
Non Patent Literature 4: Website of Fujikura Ltd.
(https://www.fujikura.co.jp/products/optical/appliedoptics/02/2051999 11309.html) retrieved on Jun. 26, 2020
Non Patent Literature 5: Website of Mitsubishi Cable Industries Ltd. (http://www.mitsubishi-cable.co.jp/ja/products/group/optical-fiber/large.html) retrieved on Jun. 26, 2020
Non Patent Literature 6: F. Gebert, M. H. Frosz, T. Weiss, Y. Wan, A. Ermolov, N. Y. Joly, P. O. Schmidt, and P. St. J. Russell, “Damage-free single-mode transmission of deep-UV light in hollow-core PCF”, Optics Express, Vol. 22, Issue 13, pp 15388, 2014.

### SUMMARY OF INVENTION

### Technical Problem

The sterilization robot is an autonomous mobile robot that emits ultraviolet light, and can automatically sterilize a wide range without human intervention by emitting ultraviolet light while moving in a room in a building such as a hospital room. However, since the robot emits high-output ultraviolet light, the device is large-scale and expensive. Therefore, the robot has a first problem that it is difficult to economically introduce the robot.

The stationary air purifier is a device that is installed on a ceiling or at a predetermined place in a room and sterilizes air in the room while circulating the air. The purifier does not directly emit ultraviolet light and does not affect the human body, and thus highly safe sterilization can be performed. However, the purifier uses a method of sterilizing the circulated indoor air, and cannot directly irradiate a place to be sterilized with ultraviolet light. Therefore, the purifier has a second problem that it has difficulty intensively sterilizing a desired site.

A portable sterilizer is a portable device equipped with an ultraviolet light source. A user can bring the device to an area to be sterilized, and can sterilize various places. However, when the user does not have skills or knowledge related to the sterilizer, it cannot be known whether or not the user has operated the sterilizer to obtain a sufficient sterilization effect at the target site, and there is a risk of the human body being affected depending on the use method. Therefore, the sterilizer has a third problem that it has difficulty securing reliability and safety.

In order to solve such problems, an ultraviolet light transmission sterilization system is considered in which an ultraviolet light source is equipped on a center side often used in architecture of an optical communication system and ultraviolet light is supplied to a remote side by optical fiber transmission. This ultraviolet light transmission sterilization system can be expected to be economical by sharing a single light source at a plurality of irradiation places, and can solve the first problem.

In addition, the ultraviolet light transmission sterilization system has flexibility to irradiate a place to be sterilized with ultraviolet light output from the tip end of the optical fiber in a pinpoint manner, and can also solve the second problem.

Furthermore, in the ultraviolet light transmission sterilization system, the output control of the light source can be performed on the center side, reliability and safety can be secured, and thus the third problem can also be solved.

However, the above-described ultraviolet light transmission sterilization system has a new problem that it is difficult for it to transmit ultraviolet light over a long distance due to optical fiber transmission characteristics of ultraviolet light. Optical fiber transmission characteristics of large-diameter fibers (Non Patent Literature 4 and 5) and hollow optical fibers (Non Patent Literature 6) in which OH groups are doped in cores have been reported. According to this report, a transmission loss of approximately 0.3 dB/m occurs in a wavelength range of 260 nm to 280 nm effective for sterilization. This value is a loss of 1500 times the transmission loss (0.0002 dB/m) in the communication wavelength band (1.5 μm band). As described above, the ultraviolet light transmission sterilization system capable of solving the first to third problems has a new problem that it is difficult for it to reduce the transmission loss from the light source (center side) to the irradiation place (remote side) of the ultraviolet light.

In order to solve the above problems, an object of the present invention is to provide a light irradiation system capable of securing economic efficiency, flexibility, reliability, and safety, and further capable of reducing a transmission loss between a center side and a remote side.

### Solution to Problem

In order to achieve the above object, the light irradiation system according to the present invention transmits light in a low-loss wavelength region from a center side through an optical fiber, and wavelength-converts the light into ultraviolet light by a nonlinear optical effect on a remote side.

Specifically, a light irradiation system according to the present invention includes:
a light source that generates propagation light having a wavelength other than a wavelength of ultraviolet light;
an irradiation unit that wavelength-converts the propagation light into ultraviolet light and irradiates a desired site with the ultraviolet light; and
an optical fiber that propagates the propagation light from the light source to the irradiation unit.

The present light irradiation system can secure economic efficiency by sharing a single light source installed on the center side with a plurality of irradiation places. The present light irradiation system can irradiate a place to be sterilized with ultraviolet light output by moving the optical fiber tip end on the remote side in a pinpoint manner, and can also secure flexibility. In addition, the present light irradiation system can secure reliability and safety by performing output control of the light source on the center side. Furthermore, in the present light irradiation system, light in a low-loss wavelength region is transmitted from the center side through the optical fiber, wavelength conversion is performed on the remote side, and thus the transmission loss between the center side and the remote side can be reduced.

Therefore, an object of the present invention is to provide a light irradiation system capable of securing economic efficiency, flexibility, reliability, and safety, and further capable of reducing a transmission loss between a center side and a remote side.

In the light irradiation system according to the present invention, the light source may generate the propagation light having one wavelength, and the irradiation unit may generate high-order harmonics from the propagation light and select the ultraviolet light from the high-order harmonics.

In the light irradiation system according to the present invention, the light source may generate the propagation light having a plurality of wavelengths, and the irradiation unit may generate a sum frequency that makes a wavelength of the ultraviolet light from the propagation light having each wavelength.

In the light irradiation system according to the present invention, it is preferable that the wavelength of the ultraviolet light be 250 nm or more and 400 nm or less.

In the light irradiation system according to the present invention, it is preferable that a feedback circuit from the irradiation unit to the light source be further provided, and that the light source adjust light intensity of the propagation light with information from the feedback circuit.

The optical fiber of the light irradiation system and an ultraviolet optical fiber that propagates the ultraviolet light from the irradiation unit to the desired site according to the present invention may be any one of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber.

The above inventions can be combined where possible.

### Advantageous Effects of Invention

The present invention provides a light irradiation system capable of securing economic efficiency, flexibility, reliability, and safety, and further capable of reducing a transmission loss between a center side and a remote side.

### BRIEF DESCRIPTION OF DRAWINGS

FIG. 1 is a diagram illustrating a light irradiation system according to the present invention.

FIG. 2 is a diagram illustrating a wavelength conversion unit of the light irradiation system according to the present invention.

FIG. 3 is a diagram illustrating an external resonator.

FIG. 4 is a diagram illustrating the light irradiation system according to the present invention.

FIG. 5 is a diagram illustrating the wavelength conversion unit of the light irradiation system according to the present invention.

FIG. 6 is a diagram illustrating the light irradiation system according to the present invention.

FIG. 7 is a diagram illustrating a cross-sectional structure of an optical fiber.

### DESCRIPTION OF EMBODIMENTS

Embodiments of the present invention will be described with reference to the accompanying drawings. The embodiments described below are examples of the present invention, and the present invention is not limited to the following embodiments. Note that components having the same reference numerals in the present specification and the drawings indicate the same components.

### First Embodiment

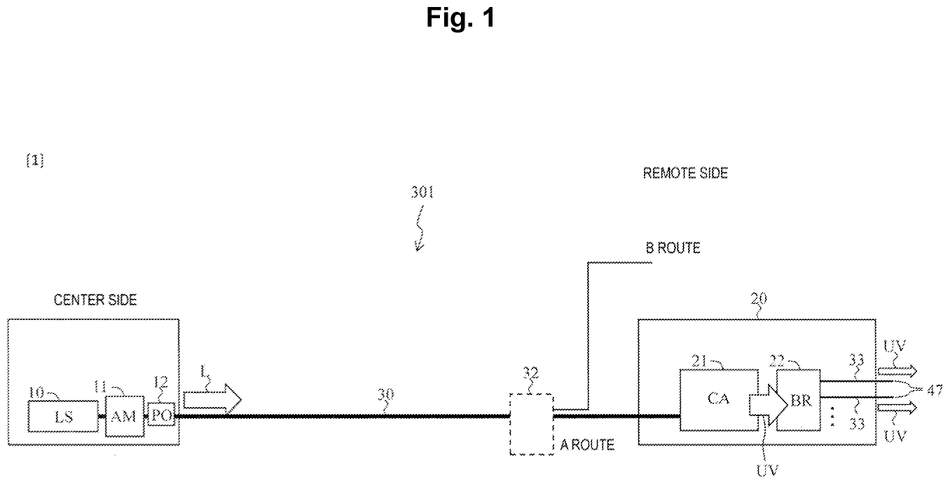

FIG. 1 is a diagram illustrating a light irradiation system 301 of the present embodiment. The light irradiation system 301 has a configuration in which one light source (fixed wavelength, variable wavelength, and pulse light) is installed on the center side, light transmitted from the light source is transmitted with a low loss through a fiber, ultraviolet light is then generated from high-order harmonics using a nonlinear optical crystal installed on the remote side, and ultraviolet light is output from the fiber tip end.

Specifically, the light irradiation system 301 includes: a light source 10 that generates propagation light L having a wavelength other than a wavelength of ultraviolet light; an irradiation unit 20 that wavelength-converts the propagation light L into ultraviolet light and irradiates a desired site with the ultraviolet light; and an optical fiber 30 that propagates the propagation light L from the light source 10 to the irradiation unit 20. In particular, the light source 10 generates the propagation light L having one wavelength, and the irradiation unit 20 generates high-order harmonics from the propagation light L and selects the ultraviolet light from the high-order harmonics.

The light source 10 is disposed on the center side. The light source 10 outputs light (propagation light L) having one wavelength (fundamental wave) other than ultraviolet light. For example, when the optical fiber 30 is a single mode optical fiber, the fundamental wave has a wavelength of 1.0 μm to 1.5 μm. Furthermore, the light source 10 may have a fixed wavelength for light to be output, or may have a variable wavelength in response to a request from the remote side. In addition, the light source 10 may output continuous light or pulse light.

The light irradiation system 301 may further include an optical amplification unit 11 that adjusts the intensity of the light output from the light source 10 on the center side. The optical amplification unit 11 amplifies the light intensity of the light having the fundamental wave in order to efficiently generate high-order harmonics. In addition, when the light intensity of the light source 10 is high, the optical amplification unit 11 may be unnecessary or may be installed on the remote side. Furthermore, the optical amplification unit 11 may adjust the amplification amount according to a request from the remote side.

The light irradiation system 301 may further include a polarization control unit 12 that adjusts the polarization of the light output from the light source 10. By adjusting the polarization of the light having the fundamental wave, efficient wavelength conversion can be performed.

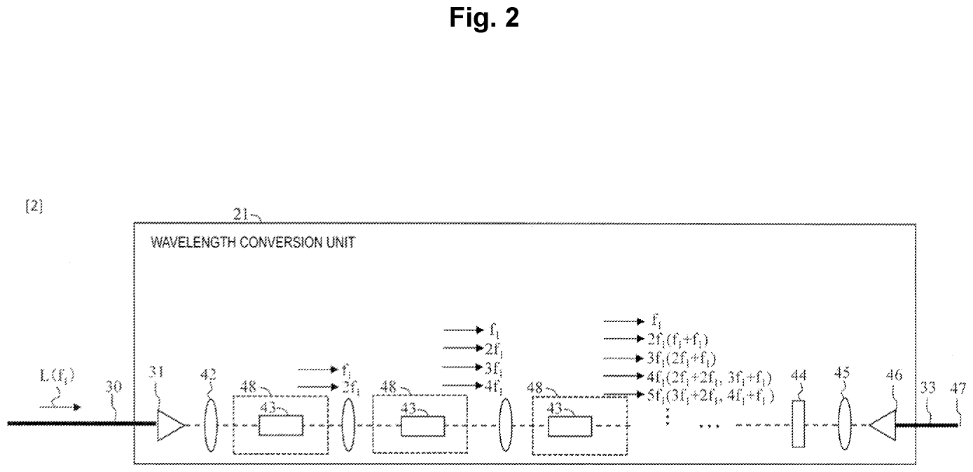

The irradiation unit 20 is disposed on the remote side near a place to be irradiated with ultraviolet light. The irradiation unit 20 includes a wavelength conversion unit 21. The wavelength conversion unit 21 performs ultraviolet conversion with high-order harmonics. FIG. 2 is a diagram illustrating a structure of the wavelength conversion unit 21. The wavelength conversion unit 21 includes a condenser lens 42, one or more nonlinear optical crystals 43, an ultraviolet light filter 44, and a condenser lens 45.

The condenser lens 42 condenses the light having the fundamental wave transmitted through the optical fiber 30 and emitted from a fiber emission unit 31, and causes the condensed light to enter the nonlinear optical crystal 43. The nonlinear optical crystal 43 generates high-order harmonics from the light having the fundamental wave. Specifically, the nonlinear optical crystal 43 is lithium triborate (LBO), β-BaB₂O₄(BBC)), or CsLiB₆O₁₀(CLBO). In the example of FIG. 2, three nonlinear optical crystals 43 are connected in series, a second harmonic wave 2f₁(f₁+f₁) having a fundamental wave f₁ is generated by the first-stage nonlinear optical crystal, a third harmonic wave 3f₁(2f₁+f₁) and a fourth harmonic wave 4f₁(2f₁+2f₁ or 3f₁+f₁) are generated by the next-stage nonlinear optical crystal, and a fifth harmonic wave 5f₁(3f₁+2f₁ or 4f₁+f₁) is generated by the third-stage nonlinear optical crystal. Note that the multistage configuration of the nonlinear optical crystal 43 in FIG. 2 is an example, and the present invention is not limited to this configuration.

The ultraviolet light filter 44 transmits ultraviolet light having a desired wavelength among the high-order harmonics generated by the nonlinear optical crystal 43. The condenser lens 45 condenses the ultraviolet light on a fiber incidence portion 46. The ultraviolet light condensed on the fiber incidence portion 46 propagates through an ultraviolet optical fiber 33 shorter than the optical fiber 30, and is emitted from an emission end 47 to a desired site.

As illustrated in FIG. 1, the ultraviolet light may be branched by a branch unit 22, and emitted from the plurality of emission ends 47 to a desired site. The branch unit 22 has a function of branching/distributing the light converted into the ultraviolet region. For example, the branch unit 22 may be an optical splitter or may be path switching by an optical switch of 1:N, or the like.

For example, when the propagation light L has a wavelength of 1064 nm (frequency f₁), the fourth harmonic wave 4f₁ (wavelength of 266 nm) among the high-order harmonics generated in the nonlinear optical crystal 43 is transmitted through the ultraviolet light filter 44. High-order harmonics other than the fourth harmonic wave 4f₁ are blocked by the ultraviolet light filter 44. The ultraviolet optical fiber 33 propagates the fourth harmonic wave 4f₁, and emits the fourth harmonic wave 4f₁ from the emission end 47 to a desired site.

In addition, when the propagation light L is 1310 nm (frequency f₁), which is often used in communication, the fifth harmonic wave 5f₁ (wavelength=262 nm) is generated by the nonlinear optical crystal 43, only the fifth harmonic wave 5f₁ is extracted as described above, and the fifth harmonic wave 5f₁ is emitted from the emission end 47 to a desired site.

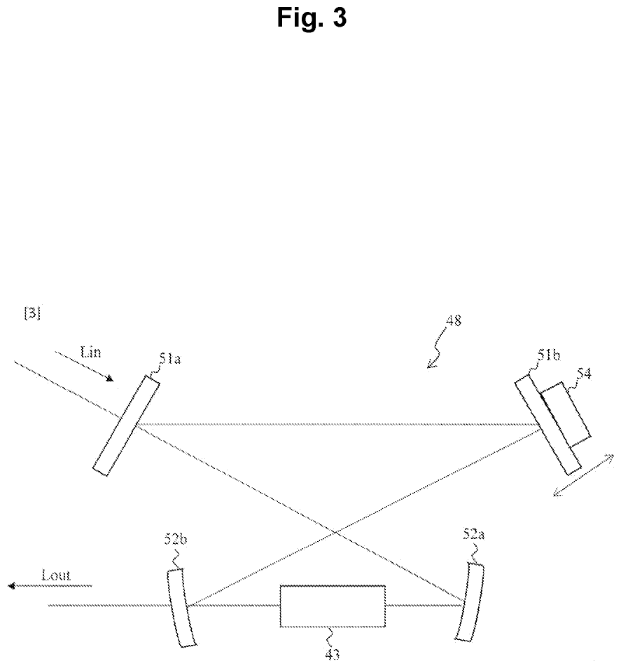

An external resonator 48 may be used to generate harmonics with high efficiency in the nonlinear optical crystal 43. The light intensity of the generated high-order harmonics becomes weak when the light is passed through the nonlinear optical crystal 43 only once. Therefore, by causing the external resonator 48 to resonate light, high-order harmonics can be generated with high efficiency. FIG. 3 is a diagram illustrating a configuration of the external resonator 48. The external resonator 48 includes a plane mirror (51a, 51b), a concave mirror (52a, 52b), and a piezoelectric element 54. The external resonator 48 efficiently generates harmonics by confining and looping the fundamental wave in the resonator.

Incident light Lin is transmitted through the plane mirror 51a, reflected by the concave mirror 52a, and incident on the nonlinear optical crystal 43. A part of the light output from the nonlinear optical crystal 43 is transmitted through the concave mirror 52b to become emitted light Lout including high-order harmonics. On the other hand, the light other than the light output from the nonlinear optical crystal 43 is reflected by the concave mirror 52b, reflected by the plane mirror 51b, the plane mirror 51a, and the concave mirror 52a, and incident on the nonlinear optical crystal 43 again. The piezoelectric element 54 adjusts the resonance frequency according to the wavelength (optical frequency) of the incident light Lin. Specifically, the oscillation frequency is adjusted to the oscillation frequency of a laser diode (LD) of the light source 10.

In addition, in the light irradiation system 301, the branch unit 32 may be disposed in the middle of the optical fiber 30 as illustrated in FIG. 1, branch the propagation light L, and supply the propagation light L to the irradiation unit 20 at another place.

### Second Embodiment

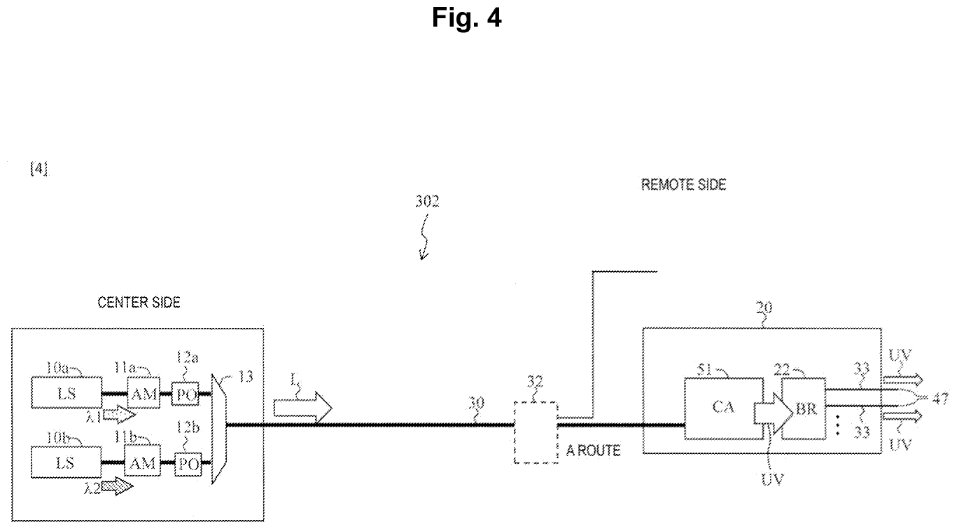

FIG. 4 is a diagram illustrating a light irradiation system 302 of the present embodiment. The light irradiation system 302 has a configuration in which two or more light sources (fixed wavelength, variable wavelength, and pulse light) is installed on the center side, light transmitted from the light source is transmitted with a low loss through a fiber, then ultraviolet light is generated by generation of sum frequency using a nonlinear optical crystal installed on the remote side, and ultraviolet light is output from the fiber tip end.

Specifically, the light irradiation system 302 includes: the light source 10 that generates the propagation light L having a wavelength other than a wavelength of ultraviolet light; the irradiation unit 20 that wavelength-converts the propagation light L into the ultraviolet light and irradiates a desired site with the ultraviolet light; and the optical fiber 30 that propagates the propagation light L from the light source 10 to the irradiation unit 20. In particular, the light source 10 generates the L propagation light having a plurality of wavelengths, and the irradiation unit 20 generates a sum frequency that makes a wavelength of ultraviolet light from the propagation light L having each wavelength.

The light source 10 is disposed on the center side. The light source 10 outputs light (propagation light L) having a plurality of wavelengths other than the ultraviolet light. In the present embodiment, a light source 10a outputs light having a wavelength λ1(optical frequency f₁), and a light source 10b outputs light having a wavelength λ2 (optical frequency f₂). For example, when the optical fiber 30 is a single mode optical fiber, λ1 and λ2 are wavelengths of 1.0 μm to 1.5 μm. Furthermore, the light source 10 may have a fixed wavelength for light to be output, or may have a variable wavelength in response to a request from the remote side. In addition, the light source 10 may output continuous light or pulse light.

The light irradiation system 302 may further include an optical amplification unit (11a, 11b) that adjusts the intensity of each light output from the light source (10a, 10b) on the center side. The optical amplification unit 11 amplifies the light intensity of light having each wavelength (21, 22) in order to efficiently generate the sum frequency. In addition, when the light intensity of the light source (10a, 10b) is high, the optical amplification unit (11a, 11b) is unnecessary. Furthermore, the optical amplification unit (11a, 11b) may adjust the amplification amount according to a request from the remote side. In addition, when the wavelength (λ1, λ2) is within the gain of the optical amplifier, one optical amplifier 11 may be installed behind the wavelength multiplexer 13 to amplify both wavelengths collectively.

The light irradiation system 302 may further include a polarization control unit (12a, 12b) that adjusts the polarization of the light output from the light source (10a, 10b). By adjusting the polarization of the light having the fundamental wave, efficient wavelength conversion can be performed.

The wavelength multiplexer 13 multiplexes light of the wavelength (λ1, λ2) and outputs the multiplexed light to the optical fiber 30 as propagation light L.

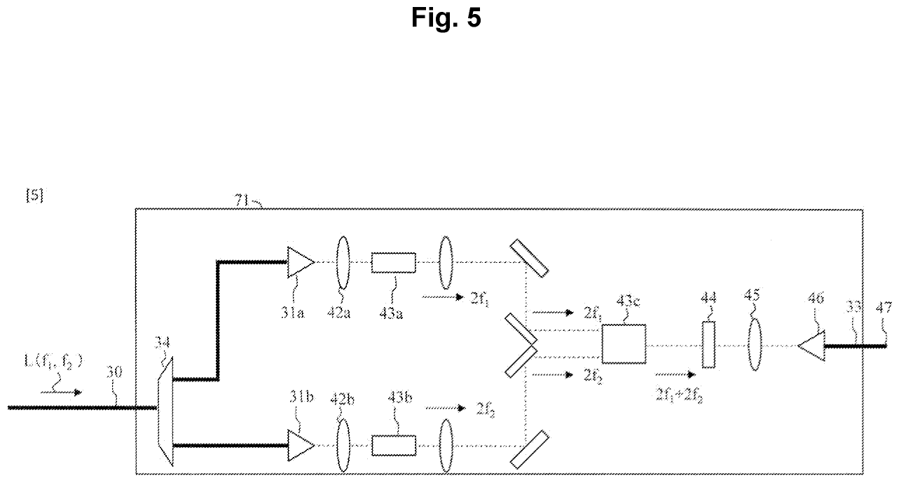

The irradiation unit 20 is disposed on the remote side near a place to be irradiated with ultraviolet light. The irradiation unit 20 includes a wavelength conversion unit 71. The wavelength conversion unit 71 performs ultraviolet conversion by the sum frequency of the wavelength λ1(optical frequency f₁) and the wavelength λ2 (optical frequency f₂). FIG. 5 is a diagram illustrating a structure of the wavelength conversion unit 71. The wavelength conversion unit 71 includes a condenser lens (42a, 42b), a nonlinear optical crystal (43a, 43b, 43c), the ultraviolet light filter 44, and the condenser lens 45.

The propagation light L transmitted through the optical fiber 30 is separated into the optical frequency f₁ and the optical frequency f₂ by a demultiplexing unit 34. Each of the condenser lenses (42a, 42b) condenses the light having the optical frequencies f₁ and f₂ emitted from the fiber emission unit (31a, 31b), and makes the emitted light incident on the nonlinear optical crystal (43a, 43b).

The nonlinear optical crystal (43a, 43b) generates high-order harmonics from incident light. Specifically, the nonlinear optical crystal 43 is lithium triborate (LBO), β-BaB₂O₄(BBO), or CsLiB₆O₁₀(CLBO). In the example of FIG. 5, the nonlinear optical crystal 43a generates the second harmonic wave 2f₁(f₁+f₁) having the optical frequency f₁, and the nonlinear optical crystal 43b generates the second harmonic wave 2f₂(f₂+f₂) of the optical frequency f₂. The light generated by each nonlinear optical crystal (43a, 43b) is incident on the nonlinear optical crystal 43c. The nonlinear optical crystal 43c generates a sum frequency (2f₁+2f₂) of the two incident light beams.

The ultraviolet light filter 44 transmits ultraviolet light having a desired wavelength from the sum frequency generated by the nonlinear optical crystal 43c. The condenser lens 45 condenses the ultraviolet light on a fiber incidence portion 46. The ultraviolet light condensed on the fiber incidence portion 46 propagates through the ultraviolet optical fiber 33 shorter than the optical fiber 30, and is emitted from the emission end 47 to a desired site.

As illustrated in FIG. 5, the ultraviolet light may be branched by the branch unit 22, and emitted from the plurality of emission ends 47 to a desired site.

For example, when the propagation light L has the wavelength λ1=1064 nm (optical frequency f₁) and the wavelength λ2=1300 nm (frequency f₂), the propagation light L is separated into the wavelength λ1(optical frequency f₁) and the wavelength λ2 (optical frequency f₂) by the demultiplexer 34 of the wavelength conversion unit 71 and emitted to the spatial system, and then the second harmonic waves 2f₁(wavelength of 532 nm) and 2f₂ (wavelength of 650 nm) are generated by the nonlinear optical crystal (43a, 43b).

The light of the second harmonic waves 2f₁ and 2f₂ is input to the nonlinear optical crystal 43c, and light having a wavelength of 292 nm is generated by generation of sum frequency (2f₁+2f₂). Light other than light having a wavelength of 292 nm is blocked by the ultraviolet light filter 44. The light having a wavelength of 292 nm is coupled to the ultraviolet optical fiber 33 and emitted from the emission end 47 to a desired site.

In addition, the light irradiation system 302 may dispose the branch unit 32 in the middle of the optical fiber 30 as illustrated in FIG. 5, branch the propagation light L, and supply the propagation light L to the irradiation unit 20 which is at another place.

In order to efficiently generate high-order harmonics in the nonlinear optical crystal (43a, 43b), the external resonator 48 described in FIG. 3 may be used. The propagation light may be directly input to the nonlinear optical crystal 43c depending on the selection of the wavelength of the ultraviolet light and the wavelength of the propagation light L.

The configuration of FIG. 5 is an example, and wavelengths of two or more light sources installed on the center side are selected, and when the desired ultraviolet light is obtained by the sum frequency generation condition (1/λ1+1/λ2=1/λ3), light having a wavelength output from the center side may be input to the nonlinear optical crystal 43c to obtain ultraviolet light.

The wavelength conversion unit 71 of the light irradiation system 302 has an advantage of being simpler in structure than the wavelength conversion unit 21 of the light irradiation system 301. Specifically, when an Nd: YAG laser with a margin in output is used as the light source 10a and a wavelength tunable laser with a relatively small output is used as the light source 10b, the fundamental wave or the harmonic of the light source 10a and the harmonic of the light source 10b are subjected to sum-frequency mixing to generate ultraviolet light, and thus there are only two stages of optical crystals that pass light from each light source in the wavelength conversion unit 71. In other words, with this configuration, the wavelength conversion unit 71 can perform wavelength conversion more efficiently than the wavelength conversion unit 21. Furthermore, the wavelength of the ultraviolet light emitted from the emission end 47 can be changed by adjusting the wavelength with the wavelength tunable laser.

### Third Embodiment

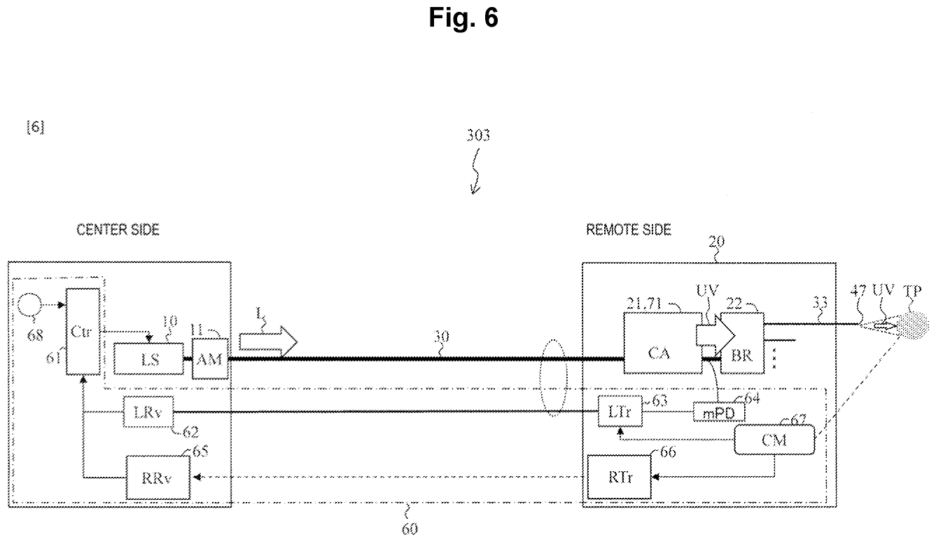

FIG. 6 is a diagram illustrating a light irradiation system 303 of the present embodiment. The light irradiation system 303 further includes a feedback circuit 60 from the irradiation unit 20 to the light source (10, 10a, 10b) with respect to the light irradiation system 301 in FIG. 1 or the light irradiation system 302 in FIG. 2, and the light source (10, 10a, 10b) adjusts the light intensity of the propagation light L with the information from the feedback circuit 60.

In the present embodiment, only parts different from the light irradiation system 301 and the light irradiation system 302 will be described.

The feedback circuit 60 is a circuit that adjusts the output of the light source 10 on the center side based on the state of the ultraviolet light on the remote side. The form of the feedback circuit 60 includes the following two examples.

(Example 1) The output of the light source 10 is adjusted based on the light intensity of ultraviolet light.

The intensity of the ultraviolet light UV generated by the wavelength conversion unit (21, 51) is detected by a monitor PD 64, and the information (output fluctuation) is fed back to the center side through an optical transmitter LTr 63. On the center side, the light source control unit 61 adjusts the light intensity output from the light source 10 or the amplification factor of the optical amplification unit 11 such that the ultraviolet light UV has a desired value based on the information through an optical receiver LVr62.

(Example 2) The output of the light source 10 is adjusted based on the image of an ultraviolet light irradiation region TP.

The ultraviolet light irradiation region TP, which is an emission destination of the ultraviolet light UV from the emission end 47, is acquired by a camera 67. The image of the ultraviolet light irradiation region TP is fed back to the center side. On the center side, the light source control unit 61 adjusts the light intensity output from the light source 10 or the amplification factor of the optical amplification unit 11 based on the image information.

Ultraviolet light (250 nm to 280 nm) having an effect on sterilization hits an absorption peak of DNA or RNA and has an effect of killing a virus. On the other hand, the ultraviolet light is also harmful to a human body (organism). Therefore, when a person appears in the ultraviolet light irradiation region TP from the image information or the emission end 47 is erroneously directed to the person himself or herself, the light source control unit 61 may perform control to immediately stop the light output from the image information. By such control, it is possible to prevent erroneous irradiation of ultraviolet light to a person, an animal, or the like (an object that is not desired to be sterilized) present in the irradiation region TP.

In addition, examples of the information transmission from the remote side to the center side include optical communication using an optical transmitter 63, an optical receiver 62, and an optical fiber 35, and wireless communication using a wireless transmitter 66 and a wireless receiver 65. In addition, in a case of optical communication, the optical fiber 30 may be bidirectional by one core using an optical circulator or a WDM multiplexer/demultiplexer instead of the optical fiber 35.

Furthermore, a timer 68 may be connected to the light source control unit 61 to control the driving time (that is, the ultraviolet light irradiation time) of the light source 10.

### Fourth Embodiment

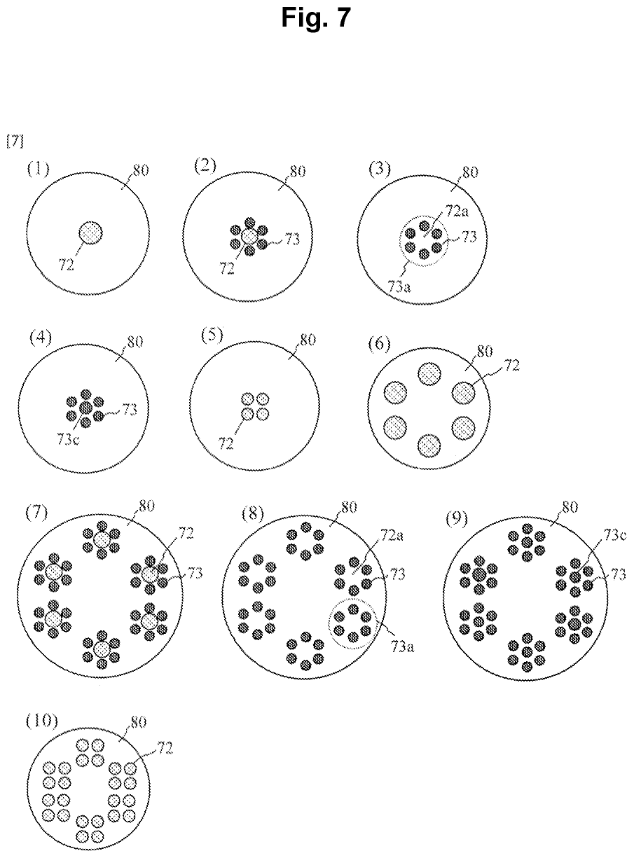

In the light irradiation system (301 to 303) described above, an optical fiber having a cross-sectional structure as illustrated in FIG. 7 can be used as the optical fiber 30 and the ultraviolet optical fiber 33.

(1) Solid Core Optical Fiber

This optical fiber has one solid core 72 having a refractive index higher than that of a clad 80, in the clad 80. “Solid” means “not hollow”. Furthermore, the solid core can also be realized by forming an annular low refractive index region in the clad.

(2) Hole Assisted Optical Fiber

The optical fiber has the solid core 72 and a plurality of holes 73 arranged on the outer periphery thereof, in the clad 80. The medium of the hole 73 is air, and the refractive index of the air is sufficiently smaller than that of quartz-based glass. Therefore, the hole assisted optical fiber has a function of returning light leaked from the core 72 by bending or the like to the core 72 again, and has a small bending loss.

(3) Hole Structure Optical Fiber

This optical fiber has a hole group 73a of the plurality of holes 73 in the clad 80, and has a refractive index effectively lower than that of a host material (glass or the like). This structure is called a photonic crystal fiber. This structure can have a structure in which a high refractive index core having a changed refractive index does not exist, and light can be confined using a region 72a surrounded by the holes 73 as an effective core region. Compared with an optical fiber having a solid core, the photonic crystal fiber can reduce the influence of absorption and scattering loss due to additives in the core, and can realize optical characteristics that cannot be realized by a solid optical fiber, such as reduction of bending loss and control of a non-linear effect.

(4) Hollow Core Optical Fiber

In this optical fiber, a core region is formed of air. Light can be confined in the core region by adopting a photonic band gap structure by a plurality of holes or an anti-resonance structure by a thin glass wire, in the clad region. This optical fiber has a small nonlinear effect, and can supply a high-power or high-energy laser.

(5) Coupling Core Type Optical Fiber

In this optical fiber, a plurality of solid cores 72 having a high refractive index are arranged close to each other in the clad 80. This optical fiber guides light between the solid cores 72 by optical wave coupling. Since the coupling core type optical fiber can disperse and transmit light by the number of cores, the power can be increased accordingly and efficient sterilization can be performed. In addition, the coupling core type optical fiber has an advantage that fiber degradation due to ultraviolet rays can be alleviated and the life can be extended.

(6) Solid Core Type Multi-core Optical Fiber

In this optical fiber, the plurality of solid cores 72 having a high refractive index are arranged apart from each other in the clad 80. This optical fiber guides light in a state where the influence of optical wave coupling can be ignored by sufficiently reducing the optical wave coupling between the solid cores 72. Therefore, the solid core type multi-core optical fiber has an advantage that each core can be treated as an independent waveguide.

(7) Hole Assisted Type Multi-core Optical Fiber

This optical fiber has a structure in which a plurality of hole structures and the core regions of (2) described above are arranged in the clad 80.

(8) Hole Structure Type Multi-core Optical Fiber

This optical fiber has a structure in which a plurality of hole structures of (3) described above are arranged in the clad 80.

(9) Hollow Core Type Multi-core Optical Fiber

This optical fiber has a structure in which a plurality of hole structures of (4) described above are arranged in the clad 80.

(10) Coupling Core Type Multi-core Optical Fiber

This optical fiber has a structure in which a plurality of coupling core structures of (5) described above are arranged in the clad 80.

### INDUSTRIAL APPLICABILITY

The light irradiation system of the present invention can be applied to an ultraviolet light sterilization system in which the wavelength of ultraviolet light to be irradiated is 250 nm or more and 400 nm or less.

### REFERENCE SIGNS LIST

10, 10a, 10b Light source
11 Optical amplification unit
12 Polarization control unit
13 Wavelength multiplexer
20 Irradiation unit
21, 71 Wavelength conversion unit
22 Branch unit
30, 35 Optical fiber
31, 31a, 31b Fiber emission unit
32 Branch unit
33 Ultraviolet optical fiber
34 Demultiplexing unit
42, 42a, 42b, 45 Condenser lens
43, 43a, 43b, 43c Nonlinear optical crystal
44 Ultraviolet light filter
46 Fiber incidence portion
47 Emission end
48 External resonator
51a, 51b Plane mirror
52a, 52b Concave mirror
54 Piezoelectric element
60 Feedback circuit
61 Light source control unit
62 Optical receiver
63 Optical transmitter
64 Monitor PD
65 Wireless receiver
66 Wireless transmitter
67 Camera
68 Timer
72 Solid core
72a Region
73 Hole
73a Hole group
80 Clad
301 to 303 Light irradiation system

1. A light irradiation system comprising:
a light source that generates propagation light having a wavelength other than a wavelength of ultraviolet light;
an irradiation unit that wavelength-converts the propagation light into the ultraviolet light and irradiates a desired site with the ultraviolet light; and
an optical fiber that propagates the propagation light from the light source to the irradiation unit.

2. The light irradiation system according to claim 1, wherein
the light source generates the propagation light having one wavelength, and the irradiation unit generates high-order harmonics from the propagation light and selects the ultraviolet light from the high-order harmonics.

3. The light irradiation system according to claim 1, wherein
the light source generates the propagation light having a plurality of wavelengths, and
the irradiation unit generates a sum frequency that makes a wavelength of the ultraviolet light from the propagation light having each wavelength.

4. The light irradiation system according to claim 1, wherein a wavelength of the ultraviolet light is 250 nm or more and 400 nm or less.

5. The light irradiation system according to claim 1, further comprising:
a feedback circuit from the irradiation unit to the light source, wherein the light source adjusts light intensity of the propagation light with information from the feedback circuit.

6. The light irradiation system according to claim 1, wherein the optical fiber is any one of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber.

7. The light irradiation system according to claim 1, further comprising:
an ultraviolet optical fiber that propagates the ultraviolet light from the irradiation unit to the desired site, wherein
the ultraviolet optical fiber is any one of a solid core optical fiber, a hole assisted optical fiber, a hole structure optical fiber, a hollow core optical fiber, a coupling core type optical fiber, a solid core type multi-core optical fiber, a hole assisted type multi-core optical fiber, a hole structure type multi-core optical fiber, a hollow core type multi-core optical fiber, and a coupling core type multi-core optical fiber.
